(19) United States
(12) Patent Application Publication
(10) Pub. No.: US 2020/0405512 A1
(43) Pub. Date: Dec. 31, 2020

(54) SYSTEMS AND METHODS FOR CONTROLLED RELEASE OF STENT BARBS

(71) Applicant: COOK MEDICAL TECHNOLOGIES LLC, Bloomington, IN (US)

(72) Inventors: Ruwan Sumanasinghe, Carmel, IN (US); Ralf Spindler, Solsberry, IN (US)

(73) Assignee: COOK MEDICAL TECHNOLOGIES LLC, BLOOMINGTON, IN (US)

(21) Appl. No.: 16/907,973

(22) Filed: Jun. 22, 2020

Related U.S. Application Data
(60) Provisional application No. 62/866,787, filed on Jun. 26, 2019.

(51) Int. Cl. A61F 2/88; A61F 2/962

(52) U.S. Cl. CPC A61F 2/88; A61F 2220/0016; A61F 2210/0014; A61F 2/962

(57) ABSTRACT

The present embodiments provide a system for controlled release of a portion of a stent. In one example, the system comprises a stent having proximal and distal regions, and a first barb coupled to the stent. A trigger wire restrains a portion of the stent in a delivery state. The system further comprises a barb release wire having proximal and distal regions, and an engagement region disposed therebetween. The distal region of the barb release wire is coupled to the trigger wire, and the engagement region of the barb release wire is disposed around a portion of the first barb to restrain the first barb in the delivery state. In one example, distal retraction of the trigger wire causes a simultaneous distal retraction of the barb release wire.

### PRIORITY CLAIM

This invention claims the benefit of priority of U.S. Provisional Application Ser. No. 62/866,787, entitled “Systems and Methods for Controlled Release of Stent Barbs,” filed Jun. 26, 2019, the disclosure of which is hereby incorporated by reference in its entirety.

### BACKGROUND

The present embodiments relate generally to apparatus and methods for treating medical conditions, and more specifically, to stents and stent-grafts for use in body vessels to treat those medical conditions.

Stents may be inserted into an anatomical vessel or duct for various purposes. Stents may maintain or restore patency in a formerly blocked or constricted passageway, for example, following a balloon angioplasty procedure. Other stents may be used for different procedures, for example, stents placed in or about a graft have been used to hold the graft in an open configuration to treat an aneurysm. Additionally, stents coupled to one or both ends of a graft may extend proximally or distally away from the graft to engage a healthy portion of a vessel wall away from a diseased portion of an aneurysm to provide endovascular graft fixation.

Stents may be either self-expanding or balloon-expandable, or they can have characteristics of both types of stents. Self-expanding stents may be delivered to a target site in a compressed configuration and subsequently expanded by removing a delivery sheath, removing trigger wires and/or releasing diameter reducing ties. With self-expanding stents, the stents expand primarily based on their own expansive force without the need for further mechanical expansion. In a stent made of a shape-memory alloy such as nitinol, the shape-memory alloy may be employed to cause the stent to return to a predetermined configuration upon removal of the sheath or other device maintaining the stent in its predeployment configuration.

Typically, the actuation of the trigger wire or other mechanism restraining a stent causes an immediate, full radial expansion of the stent, such that the stent engages an inner wall of a duct, vessel or the like. Barbs of the stent may engage the body passage, and the deployed stent may be difficult or impossible to recapture or reposition at this time.

Moreover, in some systems, barbs of a stent may extend outward during delivery within a sheath and cause an inadvertent puncturing of the sheath during delivery, or risk damaging vessels at unintended locations that may pose dangers for a patient. Further, during deployment, protruding barbs may also become inadvertently entangled with struts of the stent, which may complicate a procedure.

### SUMMARY

The present embodiments provide a system for controlled release of a portion of a stent. In one example, the system comprises a stent having proximal and distal regions, and a first barb coupled to the stent. A trigger wire restrains a portion of the stent in a delivery state. The system further comprises a barb release wire having proximal and distal regions, and an engagement region disposed therebetween. The distal region of the barb release wire is coupled to the trigger wire, and the engagement region of the barb release wire is disposed around a portion of the first barb to restrain the first barb in the delivery state. In this example, distal retraction of the trigger wire causes a simultaneous distal retraction of the barb release wire.

In another example, a system for controlled release of a portion of a stent comprises a stent having proximal and distal regions, and a first barb coupled to the stent. A trigger wire restrains a portion of the stent in a delivery state. The system comprises a barb release wire having proximal and distal regions, and an engagement region disposed therebetween. The engagement region of the barb release wire is disposed around a portion of the first barb to restrain the first barb in the delivery state. In this example, the barb release wire and the trigger wire are distally retractable in an independent manner to release the first barb and the stent portion, respectively.

In another example, a method for controlled release of a portion of a stent having proximal and distal regions and a first barb is provided. In one step, a portion of the stent is restrained in a delivery state using a trigger wire. The first barb is restrained in the delivery state using a barb release wire having proximal and distal regions, and an engagement region disposed therebetween. The engagement region of the barb release wire is disposed around a portion of the first barb to restrain the first barb. The distal region of the barb release wire is coupled to the trigger wire. Distally retracting the trigger wire causes the barb release wire to disengage from the first barb to deploy the first barb.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is an elevated perspective view of one example of a stent having at least one barb.

FIG. 2 is a partial perspective view depicting selected components of a delivery system in accordance with the present embodiments, with a portion of an outer sheath removed for illustrative purposes.

FIGS. 3A-3C are schematic side views depicting a sequence of controlled release of a portion of a stent using the system of FIG. 2.

FIGS. 4A-4B are schematic side views depicting one embodiment of a barb release wire before and after coupling to a barb.

FIGS. 5A-5B are schematic top views depicting an alternative embodiment of a barb release wire before and after coupling to a barb.

FIGS. 6A-6C are schematic side views that depict different manners in which a barb release wire may be coupled to a trigger wire.

FIGS. 7A-7C are schematic side views that depict different embodiments in which a barb release wire and a trigger wire are retracted independently from one another.

### DETAILED DESCRIPTION OF THE PREFERRED EMBODIMENTS

In the present application, the term “proximal end” is used when referring to that end of a medical device closest to the heart after placement in the human body of the patient, and may also be referred to as the inflow end (the end that receives fluid first), and the term “distal end” is used when referring to that end opposite the proximal end, or the one farther from the heart after its placement, and may also be referred to as the outflow end (that end from which fluid exits).

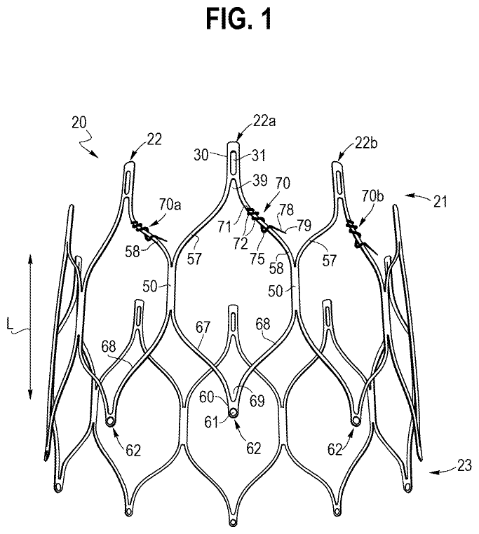

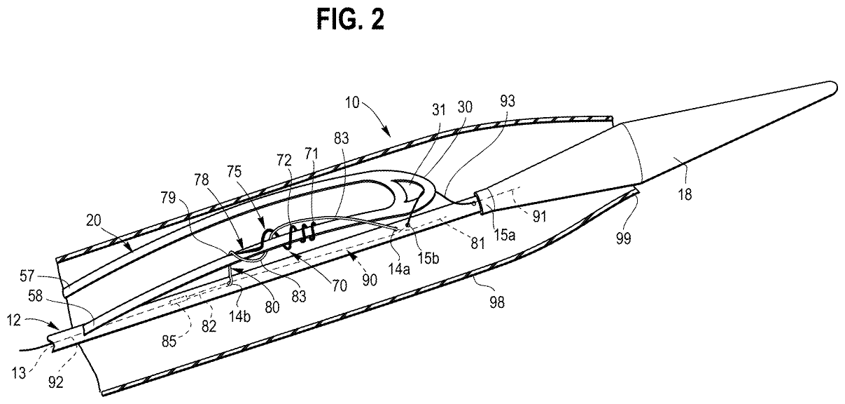

Referring to FIG. 1, a stent 20 according to a first exemplary embodiment, which may be used in conjunction with a system 10 of FIG. 2 to release the stent 20, is shown and described. In this example, the stent 20 may be manufactured from a continuous cylinder into which a pattern may be cut by a laser or by chemical etching to produce slits in the wall of the cylinder. The resulting structure may then be heat set to give it a desired final configuration. The final configuration of the stent 20 includes a shape having a series of proximal apices and a series of distal apices, as generally shown in FIG. 1. In this example, a proximal end 21 of the stent 20 may comprise multiple adjacent proximal apices 22, while a distal end 23 of the stent 20 may comprise multiple adjacent distal apices 62, as shown in FIG. 1.

The stent 20 has a reduced diameter delivery state so that it may be advanced to a target location within a vessel or duct. The stent 20 also has an expanded deployed state to apply a radially outward force upon at least a portion of a vessel or duct, e.g., to maintain patency within a passageway, or to hold open the lumen of a graft. In the expanded state, fluid flow is allowed through a central lumen of the stent 20. It will be appreciated that the struts of the stent 20 may comprise a substantially flat wire profile or may comprise a rounded profile.

The stent 20 may be manufactured from a super-elastic material. Solely by way of example, the super-elastic material may comprise a shape-memory alloy, such as a nickel titanium alloy (nitinol). If the stent 20 comprises a self-expanding material such as nitinol, the stent may be heat-set into the desired expanded state, whereby the stent 20 can assume a relaxed configuration in which it assumes the preconfigured first expanded inner diameter upon application of a certain cold or hot medium. Alternatively, the stent 20 may be made from other metals and alloys that allow the stent 20 to return to its original, expanded configuration upon deployment, without inducing a permanent strain on the material due to compression. Solely by way of example, the stent 20 may comprise other materials such as stainless steel, cobalt-chrome alloys, amorphous metals, tantalum, platinum, gold and titanium. The stent 20 also may be made from non-metallic materials, such as thermoplastics and other polymers.

In the design of FIG. 1, at least one proximal apex 22 may comprise an end region 30 having a bore 31 formed therein, wherein the bore 31 is configured to receive a trigger wire 90, as explained in further detail below. The distal apices 62 may be coupled to graft material (not shown), for example, using one or more sutures that are looped through the graft material and bores 61 formed in an end region 60 of the distal apices, as explained further below.

Referring still to FIG. 1, the stent 20 may comprise at least one strut segment disposed between the proximal and distal apices. For example, multiple angled strut segments may be disposed between a first proximal apex 22 and a corresponding distal apex 62, and identical sets of angled strut segments may be disposed between other proximal and distal apices around the circumference of the stent 20. By way of example, the first proximal apex 22 extends distally and splits into first and second angled strut segments 57 and 58, respectively, thereby forming a proximal vertex 39, as shown in FIG. 1. In a compressed state, the first and second angled strut segments 57 and 58 may be compressed such that they are substantially parallel to one another. In the expanded state shown in FIG. 1, the first and second angled strut segments 57 and 58 are disposed at an angle relative to a longitudinal axis L of the stent 20. In the expanded state, the first and second angled strut segments 57 and 58 may be disposed at an angle of about 20-60 degrees relative to the longitudinal axis L of the stent 20, as depicted in FIG. 1.

Similarly, each distal apex 62 may extend in a proximal direction and split into first and second angled strut segments 67 and 68, respectively, thereby forming a distal vertex 69. The first angled strut segments 57 and 67 of the proximal and distal apices 22 and 62, respectively, may meet with the second angled strut segments 58 and 68 of the adjacent proximal and distal apices 22 and 62, respectively, thereby forming a transition region 50. In this manner, the stent 20 may be formed into a continuous, generally cylindrical shape, as shown in FIG. 1.

Expansion of the stent 20 is at least partly provided by the angled strut segments 57, 58, 67 and 68, which may be substantially parallel to one another in a compressed state, but may tend to bow outward away from one another in the expanded state shown in FIG. 1. As noted above, the stent 20 may be formed from any suitable material, and preferably a laser-cut nitinol cannula. If manufactured from nitinol, the stent 20 may assume the expanded state shown in FIG. 1 upon removal of a delivery sheath.

Each transition region 50 may be oriented in a direction that is substantially parallel to the longitudinal axis L of the stent 20, as shown in FIG. 1. Further, each transition region 50 may comprise a larger surface area relative to the angled segments, since the transition regions are composed substantially of multiple different angled segments 57, 58, 67 and 68 meeting up at a central location.

As noted above, the distal apices 62 may be coupled to graft material, for example, using one or more sutures that are looped through the graft material and the bores 61 of the stent 20. In this manner, the stent 20 may be used as an attachment stent for endovascular graft fixation. For example, the graft material may overlap with an aneurysm to seal off fluid flow into the aneurysm, while the proximal end 22 of the stent 20 may extend in a proximal direction away from the graft material, e.g., to engage a healthy portion of a vessel wall away from a diseased portion of the aneurysm.

In accordance with one aspect, the stent 20 comprises at least one barb 70. In the embodiment of FIG. 1, the barb 70 is coupled to at least one of the angled segments 57, 58, 67 and 68 that meet up at transition regions 50. In the example of FIG. 1, different barbs 70 are coupled to different angled segments 58, although in alternative embodiments one or more barbs 70 may be coupled to selected angled segments 57, 67 or 68. It will be appreciated that the barb 70 may be coupled to other regions of the stent 20 while still achieving the advantages explained below.

In one embodiment, the barb 70 comprises a base region 71 that is secured to the angled segment 58, and further comprises a sharpened region 78 having a tip 79 that is unsecured relative to the angled segment 58. In one embodiment, the base region 71 is formed as a separate component than the angled segment 58, and comprises a plurality of coiled turns 72 that are disposed around an external surface of the angled segment 58 and secured in a suitable manner, e.g., using a solder, adhesive, weld, friction fit, a mechanical device or other means.

The barb 70 may comprise a wire coupling area 75 disposed between the base region 71 and the sharpened region 78. The wire coupling area 75 may have a bend that is wider than the coiled turns 72 and which does not engage the angled segment 58 of the stent 20, such that an open space is provided beneath the bend of the wire coupling area 75, for purposes explained further below.

FIG. 2 shows a system 10 for controlled release of a portion of the stent 20. In addition to the stent 20, the system 10 comprises a cannula 12, a barb release wire 80, a trigger wire 90, and an outer sheath 98.

The cannula 12 comprises a lumen 13 dimensioned to receive at least a portion of the barb release wire 80 and the trigger wire 90, as depicted in FIG. 2. The cannula 12 may be coupled to an atraumatic tip 18, which may subsequently transition into a smaller diameter at a proximal location to allow for atraumatic access and delivery. The trigger wire 90 may be disposed within the confines of the cannula 12, and may span the length of the cannula 12.

The trigger wire 90 has a proximal region 91, a distal region 92, and an engagement region 93 disposed therebetween. The proximal region 91 of the trigger wire 90 may be housed under the atraumatic tip 18 during delivery, as depicted in FIG. 2.

The engagement region 93 of the trigger wire 90 may be looped through the bore 31 of the proximal apex 22 of the stent 20 to restrain the stent 20 during delivery, as depicted in FIG. 2. The cannula 12 has proximal and distal trigger wire bores 15a and 15b, respectively, that permit a segment of the trigger wire 90 to extend outside of the cannula 12 to be looped around the proximal apex 22 of the stent 20, as shown in FIG. 2. The trigger wire 90 (and other trigger wires looped around other proximal apices 22 of the stent in a similar manner) may be activated by manipulating one or more handles, with optional locking features, to control deployment of the proximal end 22 of the stent 20.

The barb release wire 80 has a proximal region 81, a distal region 82, and an engagement region 83 disposed therebetween. The distal region 82 of the barb release wire 80 is coupled to the trigger wire 90, for example, using a bushing 85, as shown in FIG. 2. Although a bushing 85 is depicted, the barb release wire 80 may be coupled to the trigger wire 90 using a solder, adhesive, weld, clips, or other mechanical connections. The cannula 12 has proximal and distal release wire bores 14a and 14b, respectively, which permit the engagement region 83 of the barb release wire 80 to extend outside of the cannula 12 to be looped around the wire coupling area 75 of the barb 70, as depicted in FIG. 2.

The outer sheath 98 is longitudinally movable with respect to the other components of the system 10, and generally encircles the other components during delivery. A proximal end 99 of the outer sheath 98 may be generally flush with a portion of the atraumatic tip 18 during delivery, but may be retracted distally beyond the stent 20 to permit expansion of the stent at a target site, as explained further below.

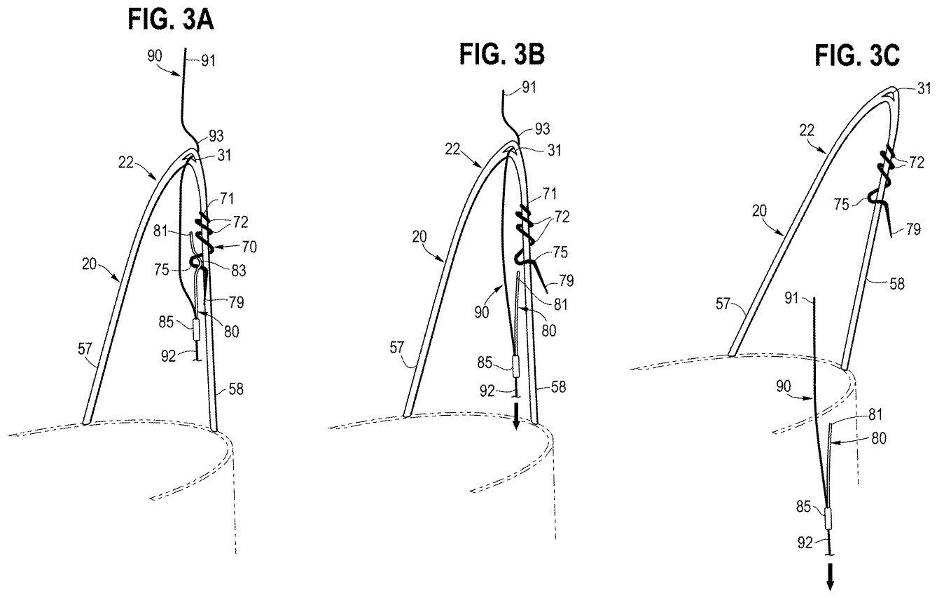

Referring to FIGS. 3A-3C, a schematic illustration of a deployment sequence of the stent 20, using the barb release wire 80 and the trigger wire 90, is shown and described. It should be noted that, in FIGS. 3A-3C, only one proximal apex 22 of the stent 20 is shown for illustrative purposes, but it will be appreciated that other apices of the stent 20 (e.g., those remaining apices shown in FIG. 1) may be deployed using the same technique for the single apex shown in FIGS. 3A-3C.

Referring to FIG. 3A, in a first delivery state, the system 10 may be advanced towards a target site within a patient's vessel or duct, in the delivery configuration of FIG. 2, over a wire guide that extends through the cannula 12. The system is advanced in an atraumatic manner guided by the atraumatic tip 18 and covered by the outer sheath 98. In this first delivery state, the trigger wire 90 is in a relatively proximal position, such that the proximal region 91 of the trigger wire 90 is housed under the atraumatic tip 18 during delivery, as depicted in FIG. 2. Further, in this first state, the engagement region 93 of the trigger wire 90 is looped through the bore 31 of the proximal apex 22 to restrain the stent 20 during delivery, as depicted in FIG. 2 and FIG. 3A.

Still further, during this first delivery state, the barb release wire 80 extends outside of the cannula 12 and is looped around the wire coupling area 75 of the barb 70, as depicted in FIG. 2 and FIG. 3A. In this example, the engagement region 83 of the barb release wire 80 directly engages and restrains the wire coupling area 75 of the barb 70, while the proximal region 81 of the barb release wire 80 extends proximal to the wire coupling area 75. In this manner, the barb release wire 80 restricts radially outward movement of the sharpened tip 79 of the barb 70.

Referring to FIG. 3B, in a second delivery state, the trigger wire 90 may be retracted distally by an operator (as indicated by the direction of the arrow), which distally retracts the barb release wire 80 that is coupled to the trigger wire 90 at the bushing 85. The retraction occurs until the proximal end 81 of the barb release wire 80 extends distal to the wire coupling area 75 of the barb 70, as depicted in FIG. 3B. At this stage, the sharpened region 78 of the barb 70 is no longer constrained and is allowed to assume its predetermined state in which the sharpened tip 79 protrudes radially outward, as shown in FIG. 3B. In this second state, the engagement region 93 of the trigger wire 90 remains looped through the bore 31 of the proximal apex 22 to continue to restrain the stent 20, as depicted in FIG. 3B.

Referring to FIG. 3C, in a third delivery state, the trigger wire 90 is further retracted distally by an operator, which causes the proximal region 91 of the trigger wire 90 to extend distally beyond the bore 31 of the proximal apex 22. At this time, when no longer constrained by the trigger wire 90, the proximal apex 22 is allowed to assume its predetermined state in which the proximal apex protrudes radially outward, as shown in FIG. 3C. Subsequently, the trigger wire 90 and other delivery components may be removed from a patient's body.

Advantageously, the system 10 of FIGS. 1-3 permits a more controlled deployment of the barbs 70 of the stent 20. Unlike prior systems in which barbs expand radially outward upon retraction of a sheath, the barbs 70 of present system 10 will not expand radially outward merely based on retraction of the sheath 98. Instead, the barb release wire 80 restrains the barb 70 until a time at which deployment of the barb 70 is in fact desirable, which may be after retraction of the sheath 98. In this manner, the sheath 98 may expose the stent 20, and optionally a portion of graft material if coupled to the stent 20, such that an operator can better visualize the specific location of components without sheath obstruction, and further permit a degree of expansion of the stent 20 prior to committing to full deployment of the barbs 70 and the stent 20. Better conformability of the stent 20, along with any graft coupled to the stent, may be achieved in a body vessel because the barbs 70 are significantly less likely to prematurely engage with the body vessel when restrained using the barb release wire 80.

As a further advantage, the barbs 70 are proactively restrained by an independent mechanism within the sheath 98, and not merely by the sheath 98 itself. In particular, by having the barb release wire 80 independently restrain the barbs 70 when inside of the sheath 98, the barbs 70 may be less likely to inadvertently pierce through the sheath 98. Such inadvertent piercing of the sheath 98 may pose a danger to the patient as a vessel wall may become punctured when advancing the sheath 98 in a pierced condition. The barb release wire 80 of the present embodiments significantly reduces such risk, as well as the risk of physician injuries due to premature deployment of the barbs. Moreover, the present embodiments reduce the risk of barb interferement with the sheath 98 while the sheath 98 is being withdrawn.

As a further advantage, the barb release wire 80 makes it less likely for the barbs 70 to become entangled with segments of the stent 20. In prior devices, premature barb deployment could yield entanglement with stent struts, which could render the operation difficult or impossible to complete, but such risk is significantly reduced in the present embodiments due to the high degree of barb control.

As yet a further advantage, when the stent 20 comprises multiple barbs 70, different barbs may be released at different times. Referring back to FIG. 1, the stent 20 may comprise first and second barbs 70a and 70b that are secured to different apices. It may be beneficial to deploy the first barb 70a before the second barb 70b to improve orientation of the stent 20. In this embodiment, two different barb release wires 80 may be provided, where one is coupled to the first barb 70a in the manner shown in FIGS. 3A-3C and the other is coupled to the second barb 70b in the same manner.

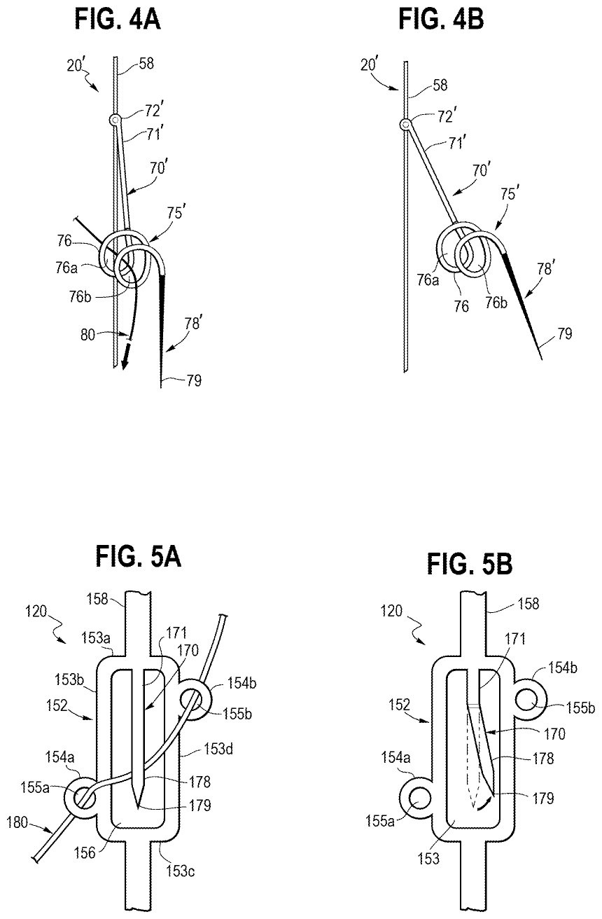

Referring to FIGS. 4A-4B, an alternative barb 70′ of a stent 20′ is shown in restrained and expanded states, respectively. The barb 70′ comprises a wire coupling area 75′ disposed between a base region 71′ and a sharpened region 78′. In this embodiment, the base region 71′ does not comprise coiled turns that encircle the angled segment 58 of the stent 20′, as in the embodiment of FIGS. 1-3, but rather comprises a generally straight segment that is integrally formed with the angled segment 58 or alternatively is secured to the angled segment 58 at coupling location 72′, e.g., using a solder, adhesive, weld, or mechanical coupling.

The wire coupling area 75′ comprises at least one coiled turn 76, and in the embodiment of FIGS. 4A-4B comprises two coiled turns 76a and 76b. In the restrained state, the barb release wire 80 extends through the open space within the coiled turns 76a and 76b in a relatively taut manner, thereby restraining the sharpened region 78′ of the barb 70′ radially inward towards the stent 20′, as shown in FIG. 4A. When the barb release wire 80 is retracted distally, such that it is no longer disposed within the coiled turns 76a and 76b of the barb 70′, then the barb 70′ is permitted to assume its relaxed or expanded state as shown in FIG. 4B.

Referring to FIGS. 5A-5B, an alternative stent 120 comprises at least one strut region 158 having a barb housing 152. In this example, the barb housing 152 generally comprises four sides 153a through 153d, with an open space 156 in the interior. A base portion 171 of a barb 170 is secured to the side 153a of the barb housing 152, and extends over a portion of the open space 156 towards the opposing side 153c of the barb housing 152. A sharpened region 178 with tip 179 of the barb 170 terminates prior to the side 153c of the barb housing 152.

A first guide 154a having a channel 155a is disposed at the side 153b of the barb housing 152, while a second guide 154b having a channel 155b is disposed at the opposing side 153d of the barb housing 152, as shown in FIGS. 5A-5B. The first and second guides 154a and 154b may be in the form of eyelets, and may be formed integrally with the barb housing 152, or externally coupled to the barb housing 152, e.g., soldered to the opposing sides. In this example, the guide 154b is depicted as being vertically above the other guide 154a, or alternatively stated closer to side 153a, but in alternative embodiments the guides 154a and 154b may be at substantially the same height.

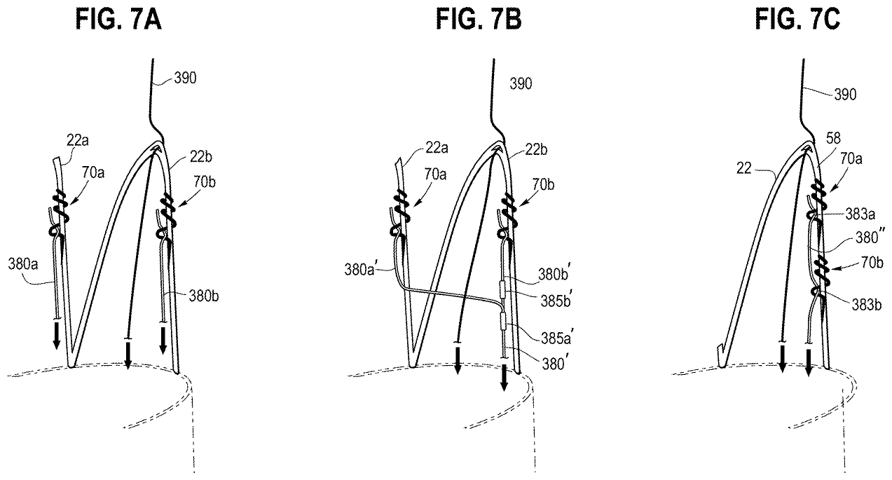

In the restrained state, the barb release wire 180 extends through the channel 155a of the first guide 154a, then under the barb 170 in a relatively taut manner, and then through the channel 155b of the second guide 154b, as shown in FIG. 5A. In this manner, the barb 170 is prevented from moving towards its relaxed state, which is into the page in the depiction of FIG. 5A. When the barb release wire 180 is retracted distally, such that it is no longer disposed within the first and second guides 154a and 154b, then the barb 170 is permitted to assume its relaxed or expanded state, as shown in FIG. 5B. It should be noted that, in the example of FIGS. 5A-5B, a distal region of the barb release wire 180 may be secured to a trigger wire in the manner described above in FIGS. 1-3, or alternatively may be an independent wire spanning the length of a delivery system and actuated separately from the trigger wire, similar to the alternative embodiments of FIGS. 7A-7C below that describe independent trigger wire and barb release wires that are not coupled to each other.

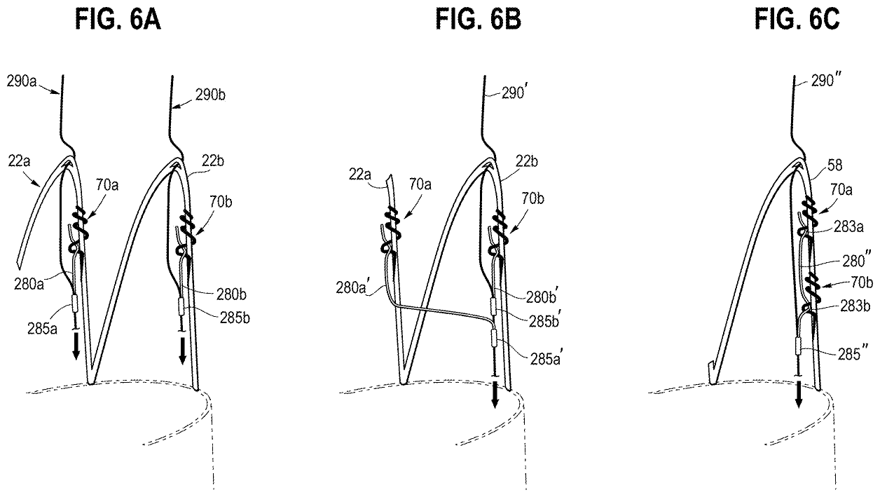

Referring to FIGS. 6A-6C, three variations in which the barb release wires may be coupled to trigger wires to achieve the functionality and advantages noted above are shown and described. In FIG. 6A, the system comprises the same principles of FIGS. 3A-3C, and shows a first barb release wire 280a being coupled to a first trigger wire 290a at a bushing 285a, and a second barb release wire 280b being coupled to a second trigger wire 290b at a bushing 285b. Each trigger wire 290a and 290b spans the full length of the lumen 13 of the cannula 12 of FIG. 2, and engages an apex 22 of the stent 20 as depicted in FIG. 6A. Distal retraction of the first trigger wire 290a and its associated barb release wire 280a releases a first barb 70a and then the first apex 22a in the manner described above in FIGS. 2-3, while distal retraction of the second trigger wire 290b and its associated barb release wire 280b releases a second barb 70b and then the second apex 22b. Advantageously, in this manner, different barbs may be released at different times, which may improve orientation of the stent 20 during delivery.

In FIG. 6B, a single trigger wire 290′ is coupled to two different barb release wires 280a′ and 280b′ at locations 285a′ and 285b′, respectively. The couplings may be by bushings or other mechanisms listed above. The barb release wire 280a′ extends in a direction to restrain the first barb 70a, while the barb release wire 280b′ extends in a direction to restrain the second barb 70b, as shown in FIG. 6B. During use, distal retraction of the trigger wire 290′ simultaneously retracts both release wires 280a′ and 280b′, which may deploy the barbs 70a and 70b at substantially the same time. The embodiment of FIG. 6B may be beneficial because it may reduce the number of trigger wires to less than one per proximal apex, while still providing a barb release wire to every apex, and such reduction in trigger wires may reduce the overall delivery profile.

In FIG. 6C, a single trigger wire 290″ is secured to a single barb release wire 280″ at location 285″, but in this embodiment the barb release wire 280″ is configured to engage a plurality of barbs 70a and 70b. In one example, the barbs 70a and 70b are coupled to an angled segment 58 of the stent 20 in an axially aligned manner, e.g., where the barb 70a is disposed proximal to the barb 70b. The barb release wire 280″ may comprise a first region 283a that directly engages and restrains the wire coupling area 75 of the barb 70a, and further comprises a second region 283b that directly engages and restrains the wire coupling area 75 of the barb 70b, as depicted in FIG. 6C. During use, distal retraction of trigger wire 290″ simultaneously retracts the barb release wire 280″ to first release the barb 70a, and upon further distal retraction, to subsequently release the barb 70b. The embodiment of FIG. 6C may be beneficial because it may reduce the number of barb release wires to less than one per barb, while still providing a controlled barb release ability to every barb. Such reduction in barb release wires may reduce the overall delivery profile, and allow for a sequential release of nearby barbs.

Referring to FIGS. 7A-7C, three variations in which the barb release wires may be uncoupled to the trigger wires, while still achieving key functionality and advantages noted above, are shown and described. In FIG. 7A, a trigger wire 390 spans the full length of the lumen 13 of the cannula 12 of FIG. 2, and two different barb release wires 380a and 380b also span the full length of the lumen 13 of the cannula 12. The first barb release wire 380a directly engages and restrains a first barb 70a on a first apex 22a of the stent, while the second barb release wire 380b directly engages and restrains a second barb 70b on a second apex 22b of the stent. During use, the first and second barb release wires 380a and 380b may be independently retracted in a distal direction to permit expansion of their associated barbs 70a and 70b, and at an independent time the trigger wire 390 may be distally retracted to allow full radial expansion of the apices 22a and 22b. Advantageously, in this manner, complete independent control is provided to deploy each barb independently, and also to deploy each apex independently.

In FIG. 7B, a trigger wire 390 spans the full length of the lumen 13 of the cannula 12 of FIG. 2, and a separate barb release wire 380′ is coupled to two different barb release wire segments 380a′ and 380b′ at locations 385a′ and 385b′, respectively. The couplings may be by bushings or other mechanisms listed above. The barb release wire segment 380a′ extends in a direction to restrain the first barb 70a, while the barb release wire segment 380b′ extends in a direction to restrain the second barb 70b, as shown in FIG. 7B. During use, distal retraction of the barb release wire 380′ simultaneously retracts both release wire segments 380a′ and 380b′, which may deploy the barbs 70a and 70b at substantially the same time. The embodiment of FIG. 7B may be beneficial because it may reduce the number of barb release wires that span the full length of the delivery system to less than one per proximal apex, while still providing a trigger wire to every apex, and such reduction in barb release wires may reduce the overall delivery profile.

In FIG. 7C, a trigger wire 390 spans the full length of the lumen 13 of the cannula 12 of FIG. 2, and a separate barb release wire 380″ also spans the full length of the cannula 12 and is configured to engage a plurality of barbs 70a and 70b. In this example, as in FIG. 6C, the barbs 70a and 70b may be coupled to an angled segment 58 of the stent 20 in an axially aligned manner, e.g., where the barb 70a is disposed proximal to the barb 70b. The barb release wire 380″ may comprise a first region 383a that directly engages and restrains the wire coupling area 75 of the barb 70a, and further comprises a second region 383b that directly engages and restrains the wire coupling area 75 of the barb 70b, as depicted in FIG. 7C. During use, the barb release wire 380″ may be independently retracted in a distal direction to permit expansion of their associated barbs 70a and 70b, and at an independent time the trigger wire 390 may be distally retracted to allow full radial expansion of the apex 22. Advantageously, such embodiment may reduce the number of barb release wires to less than one per barb, while still providing a controlled barb release ability to every barb.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A system for controlled release of a portion of a stent, the system comprising:
a stent having proximal and distal regions;
a first barb coupled to the stent;
a trigger wire that restrains a portion of the stent in a delivery state; and
a barb release wire having proximal and distal regions, and an engagement region disposed therebetween,
wherein the distal region of the barb release wire is coupled to the trigger wire, and the engagement region of the barb release wire is disposed around a portion of the first barb to restrain the first barb in the delivery state,
wherein distal retraction of the trigger wire causes a simultaneous distal retraction of the barb release wire.

2. The system of claim 1, wherein the barb release wire spans a shorter axial length than the trigger wire.

3. The system of claim 1, wherein the trigger wire restrains at least one proximal apex of the stent in the delivery state.

4. The system of claim 3, wherein the at least one proximal apex comprises an end region having a bore formed therein, wherein the trigger wire extends through the bore in the delivery state.

5. The system of claim 1, wherein the first barb comprises a proximal segment having a base region coupled to the stent, a distal segment having a sharpened tip, and a wire coupling area disposed between the proximal and distal segments, wherein the barb release wire is disposed around the wire coupling area of the first barb in the delivery state.

6. The system of claim 5, wherein the base region of the first barb comprises a plurality of coiled turns disposed around an external surface of the stent, and wherein the wire coupling area of the stent comprises a segment wider than the plurality of coiled turns and does not engage the stent.

7. The system of claim 1, wherein the stent comprises a barb housing comprising an open space that houses the first barb, and further comprises first and second opposing guides, wherein the barb release wire is looped through the first guide, over the first barb, and through the second guide to restrain the first barb in the delivery state.

8. The system of claim 1, further comprising a cannula comprising a lumen dimensioned to receive at least a portion of the barb release wire and the trigger wire.

9. The system of claim 8, wherein the cannula comprises proximal and distal trigger wire bores, wherein a segment of the trigger wire extends outside of the cannula between the proximal and distal trigger wire bores to restrain the stent in the delivery state.

10. The system of claim 9, wherein the cannula comprises proximal and distal release wire bores, wherein the engagement region of the barb release wire extends outside of the cannula between the proximal and distal release wire bores to restrain the first barb in the delivery state.

11. The system of claim 1, wherein the barb release wire is coupled to the trigger wire using a bushing.

12. The system of claim 1, further comprising an outer sheath extending over the stent, the first barb, the trigger wire and the barb release wire in the delivery state, wherein distal retraction of the outer sheath exposes the stent in a release state.

13. The system of claim 1, further comprising a second barb coupled to the stent, wherein the first and second barbs are selectively released at different times.

14. The system of claim 13, wherein the barb release wire is disposed around each of the first and second barbs in the delivery state.

15. A system for controlled release of a portion of a stent, the system comprising:
a stent having proximal and distal regions;
a first barb coupled to the stent;
a trigger wire that restrains a portion of the stent in a delivery state; and
a barb release wire having proximal and distal regions, and an engagement region disposed therebetween,
wherein the engagement region of the barb release wire is disposed around a portion of the first barb to restrain the first barb in the delivery state,
wherein the barb release wire and the trigger wire are distally retractable in an independent manner to release the first barb and the stent portion, respectively.

16. The system of claim 15, wherein the first barb comprises a proximal segment having a base region coupled to the stent, a distal segment having a sharpened tip, and a wire coupling area disposed between the proximal and distal segments, wherein the barb release wire is disposed around the wire coupling area of the first barb in the delivery state.

17. The system of claim 15, further comprising a second barb coupled to the stent, wherein the barb release wire is disposed around each of the first and second barbs in the delivery state.

18. A method for controlled release of a portion of a stent, the stent having proximal and distal regions and a first barb coupled to the stent, the method comprising:
restraining a portion of the stent in the delivery state using a trigger wire;
restraining the first barb in the delivery state using a barb release wire having proximal and distal regions, and an engagement region disposed therebetween,
wherein the engagement region of the barb release wire is disposed around a portion of the first barb to restrain the first barb,
wherein the distal region of the barb release wire is coupled to the trigger wire; and
distally retracting the trigger wire to cause the barb release wire to disengage from the first barb to deploy the first barb.

19. The method of claim 18 further comprising distally retracting the trigger wire to subsequently deploy the portion of the stent.

20. The method of claim 18, wherein a second barb is coupled to the stent, the method further comprising using the barb release wire to restrain both the first and second barbs in the delivery state, and retracting the barb release wire to sequentially deploy the first barb and then the second barb.
